(19) United States
(12) Patent Application Publication
(10) Pub. No.: US 2026/0354958 A1
(43) Pub. Date: Nov. 12, 2026

(54) PESSARY DEVICES AND METHODS

(71) Applicant: LEVAIRA, INC., WELLESLEY, MA (US)

(72) Inventor: ROBERT SLAZAS, PALMETTO BAY, FL (US)

(73) Assignee: LEVAIRA, INC., WELLESLEY, MA (US)

(21) Appl. No.: 19/201,201

(22) Filed: May 7, 2025

(51) Int. Cl. A61F 2/00; A61F 6/08; A61L 31/02; A61L 31/06

(52) U.S. Cl. CPC A61F 2/0022; A61F 6/08; A61L 31/022; A61L 31/06; A61F 2210/0014; A61F 2230/0008; A61L 2400/16

(57) ABSTRACT

An intra-vaginal pessary device includes an outer ring including a first opening. The outer ring is configured to engage vaginal surface(s) of a patient. The pessary device can include a shape memory component disposed within the outer ring, a net disposed within the first opening, a third opening disposed within the first opening, a distal knob, and a cutout. The third opening is configured to aid in removing the pessary device from the patient's vaginal canal. The distal knob is configured to provide urethral support, and the cutout is disposed along the outer ring and is configured to engage levator muscle(s) of the patient. The pessary device is movable between a first position in a compressed state and a second position in a deployed state such that the pessary device can be delivered into the patient's vaginal canal when in the compressed state and then expand into the deployed state.

### FIELD

The present disclosure generally relates to pessary devices and methods of using the same. More specifically, certain embodiments relate to intra-vaginal pessary devices configured to engage with one or more vaginal surfaces and levator muscles of a patient.

### BACKGROUND

Pessary devices can be used to address stress urinary incontinence, pelvic floor disorders, and pelvic organ prolapse in adult females. These devices are typically inserted into a patient's vaginal canal to provide structure for the surrounding organs. Existing devices must typically be checked either by the patient or a physician about every three months.

Accordingly, alternative pessary devices and methods would be useful.

### SUMMARY

The present disclosure is directed to pessary devices and methods of using the same.

An example pessary device is disclosed. The pessary device can be an intra-vaginal pessary device that has an outer ring having an elliptical shape and including a first opening. At least a portion of the outer ring is configured to engage one or more vaginal surfaces of a patient. The pessary device can further include a shape memory component disposed within the outer ring and internal to the pessary device. The pessary device can further include a net disposed within the first opening of the outer ring, the net itself having one or more second openings. The pessary device can further include a third opening disposed within the first opening and distal to the net, the third opening configured to aid in at least removing the pessary device from the patient's vaginal canal. The pessary device can further include a distal knob configured to provide urethral support. The pessary device can further include a cutout disposed along the outer ring and configured to engage one or more levator muscles of the patient. The pessary device is movable between a first position in a compressed state and a second position in a deployed state such that the pessary device can be delivered into the patient's vaginal canal and placed transverse to the patient's vaginal canal when in the compressed state and then expand into the deployed state.

Another example pessary device is disclosed. The pessary device can be an intra-vaginal pessary device that has an outer ring having an elliptical shape and including a first opening. At least a portion of the outer ring is configured to engage one or more vaginal surfaces of a patient. The pessary device can further include a shape memory component disposed within the outer ring and internal to the pessary device. The pessary device can further include a second opening disposed within the first opening. The second opening is configured to aid in at least removing the pessary device from the patient's vaginal canal. The pessary device can further include a cutout disposed along the outer ring and configured to engage one or more levator muscles of the patient. The pessary device is movable between a first position in a compressed state and a second position in a deployed state such that the pessary device can be placed transversely into the patient's vaginal canal when in the compressed state and then expand into the deployed state.

Another example pessary device is disclosed. The pessary device can be an intra-vaginal pessary device that has an outer ring configured to engage one or more vaginal surfaces of a patient. The pessary device can further include a cutout disposed along the outer ring and configured to engage one or more levator muscles of the patient when the pessary device is placed in a deployed state within the patient's vaginal canal. The pessary device is movable between a first position in a compressed state and a second position in a deployed state such that the pessary device can be placed within the patient's vaginal canal.

### BRIEF DESCRIPTION OF THE DRAWINGS

The above and further aspects of this disclosure are further discussed with reference to the following description in conjunction with the accompanying drawings, in which like numerals indicate like structural elements and features in various figures. The drawings are not necessarily to scale, emphasis instead being placed upon illustrating principles of the disclosure. The figures depict one or more implementations of the inventive devices, by way of example only, not by way of limitation.

FIG. 1 is a perspective view of an example pessary device, according to aspects of the present disclosure.

FIG. 2 is a top view of the example pessary device of FIG. 1, according to aspects of the present disclosure.

FIG. 3 is a bottom view of the example pessary device of FIG. 1, according to aspects of the present disclosure.

FIG. 4 is a right side perspective view of the example pessary device of FIG. 1, according to aspects of the present disclosure.

FIG. 5 is a right side view of the example pessary device of FIG. 1, according to aspects of the present disclosure.

FIG. 6 is a back perspective view of the example pessary device of FIG. 1, according to aspects of the present disclosure.

FIG. 7 is a back view of the example pessary device of FIG. 1, according to aspects of the present disclosure.

FIG. 8 is a left side perspective view of the example pessary device of FIG. 1, according to aspects of the present disclosure.

FIG. 9 is a left side view of the example pessary device of FIG. 1, according to aspects of the present disclosure.

FIG. 10 is a front view of the example pessary device of FIG. 1, according to aspects of the present disclosure.

FIG. 11 is a bottom perspective view of the example pessary device of FIG. 1, according to aspects of the present disclosure.

FIG. 12 is a top perspective view of the example pessary device of FIG. 1, according to aspects of the present disclosure.

FIG. 13 is a perspective view of an example component of the example pessary device of FIG. 1, according to aspects of the present disclosure.

FIG. 14 is a top view of the example component of FIG. 13, according to aspects of the present disclosure.

FIG. 15 is a right side perspective view of the example component of FIG. 13, according to aspects of the present disclosure.

FIG. 16 is a right side view of the example component of FIG. 13, according to aspects of the present disclosure.

FIG. 17 is a perspective view of an example pessary device, according to aspects of the present disclosure.

FIG. 18 is a right side view of the example pessary device of FIG. 17, according to aspects of the present disclosure.

FIG. 19 is a back perspective view of the example pessary device of FIG. 17, according to aspects of the present disclosure.

FIG. 20 is a back view of the example pessary device of FIG. 17, according to aspects of the present disclosure.

FIG. 21 is a bottom perspective view of the example pessary device of FIG. 17, according to aspects of the present disclosure.

FIG. 22A is a top view of an example pessary device in a compressed state, according to aspects of the present disclosure.

FIG. 22B is a top view of an example pessary device in a compressed state, according to aspects of the present disclosure.

FIG. 23 is an illustration of an existing pessary device inserted in a vaginal canal.

FIG. 24 is an illustration of an example pessary device inserted in a vaginal canal, according to aspects of the present disclosure.

FIG. 25 is an illustration of an example pessary device inserted in a vaginal canal, according to aspects of the present disclosure.

### DETAILED DESCRIPTION

Existing pessary devices used to address stress urinary incontinence (SUI), pelvic floor disorders, and/or pelvic organ prolapse (POP) can be difficult to handle (e.g., due to their bulk), and can cause side effects. Additionally, existing pessary devices are prone to spontaneous displacement, migration, and/or rotation, rendering them less effective, less consistent in providing support (e.g., urethral and vaginal support), and more likely to be expelled from proper placement in the vaginal canal. A large proportion of patients who use existing pessary devices end up discontinuing their use due to these and other challenges.

The example pessary devices described herein aid in overcoming these and other challenges with existing pessary devices through the incorporation of specific components that uniquely interact with surfaces of the patient's vaginal canal and/or the patient's levator muscles. Such interaction provides improved usability, comfort, and effectiveness in treating certain patient conditions. The disclosed pessary devices can also be made in a variety of sizes to accommodate different patient sizes, and can incorporate different materials (e.g., silicone).

Reference will now be made in detail to example embodiments of the disclosed technology that are illustrated in the accompanying drawings and disclosed herein. Wherever convenient, the same reference numbers will be used throughout the drawings to refer to the same or like parts.

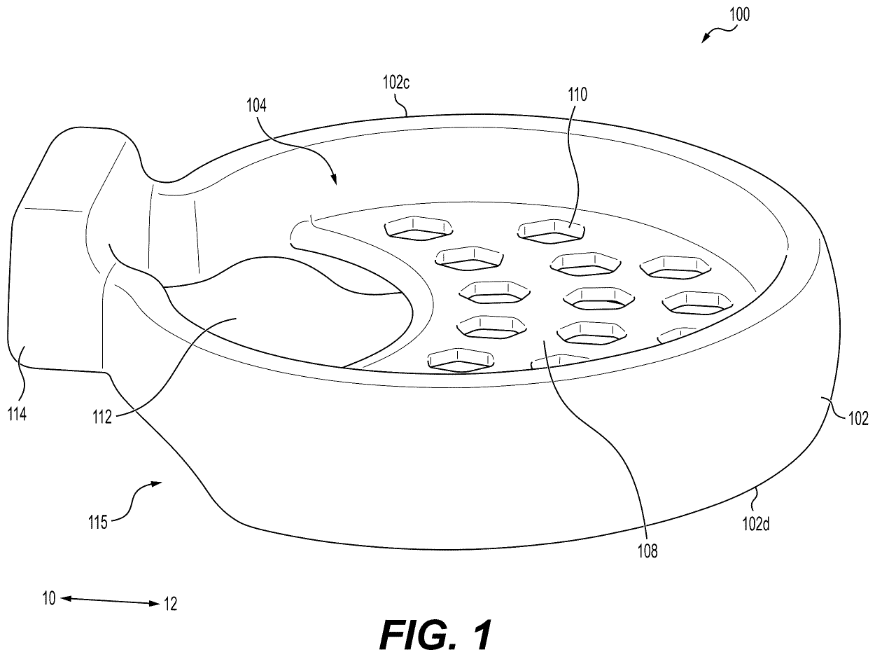

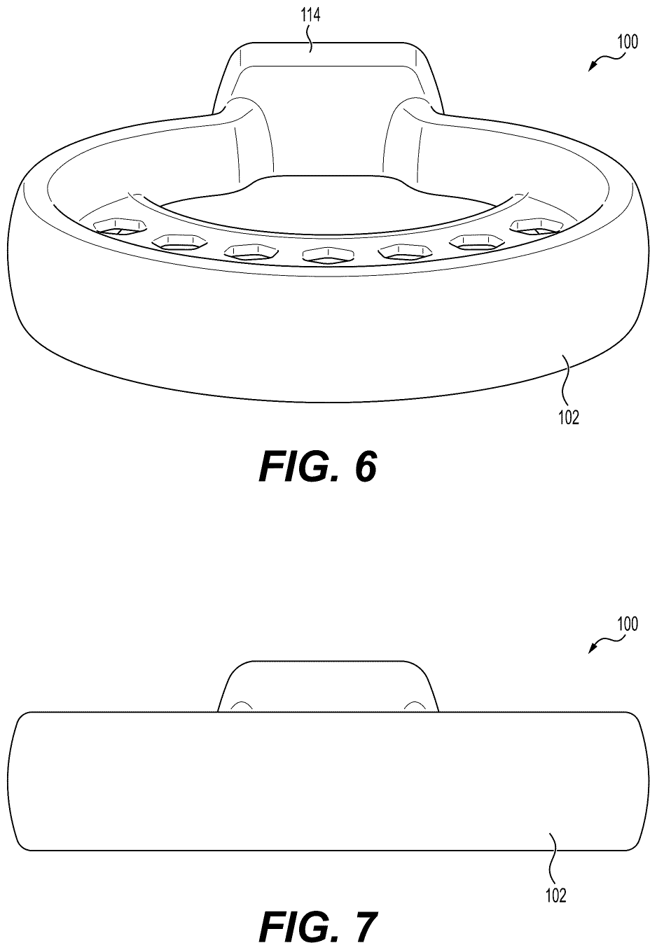

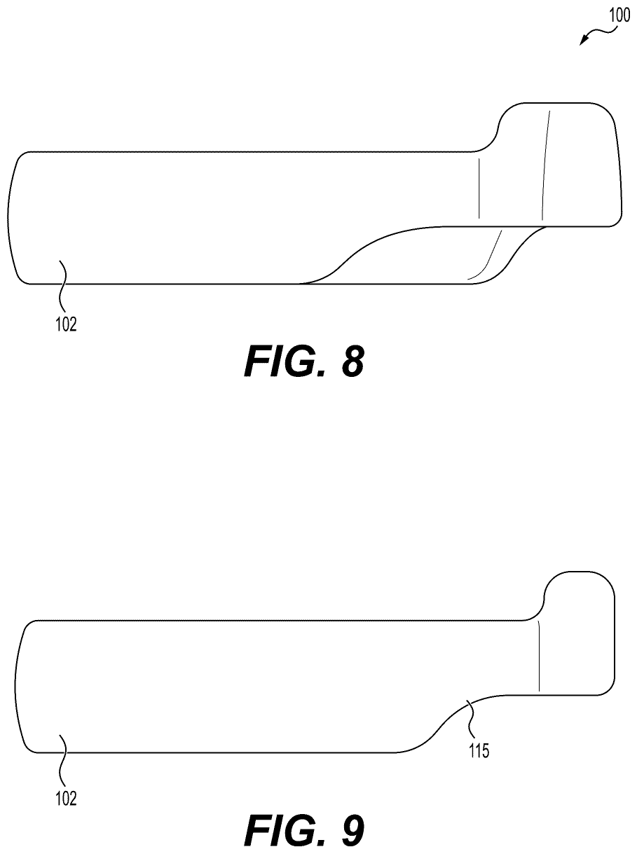

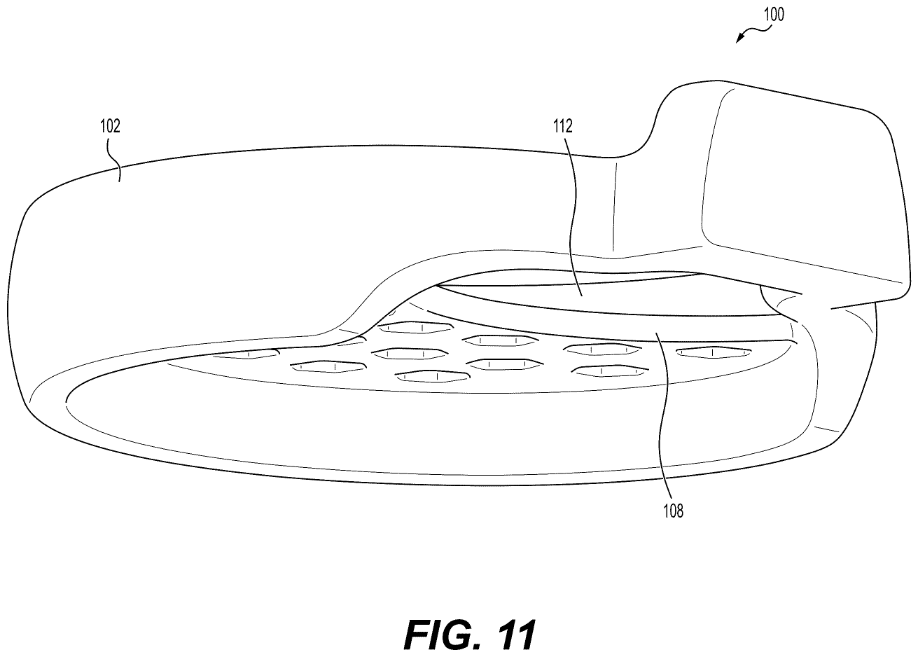

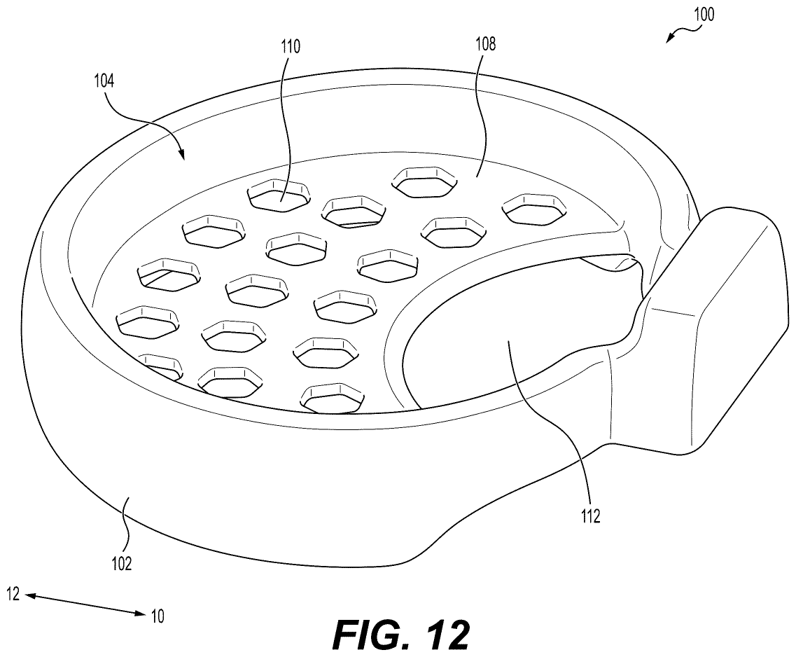

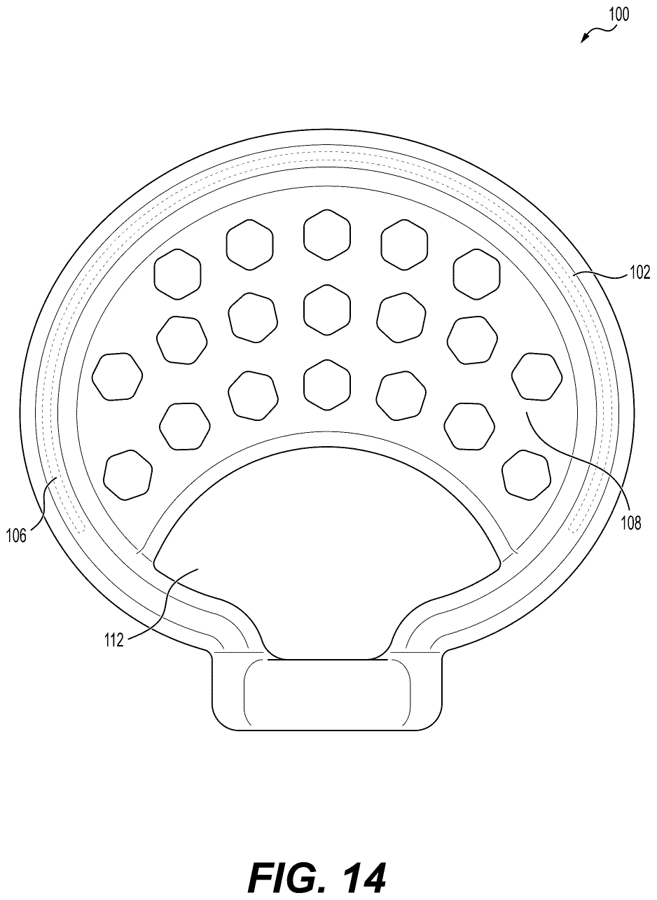

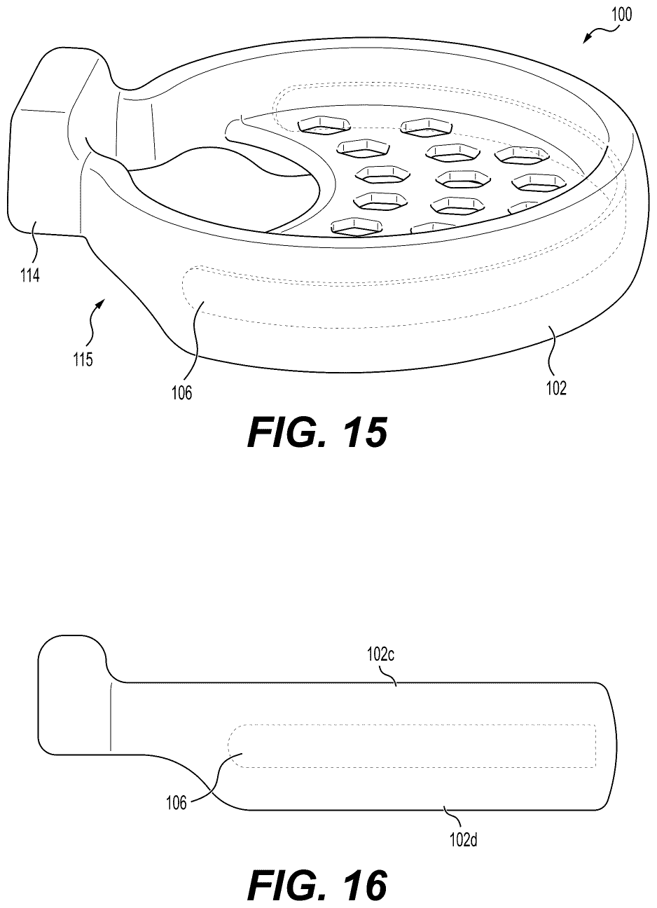

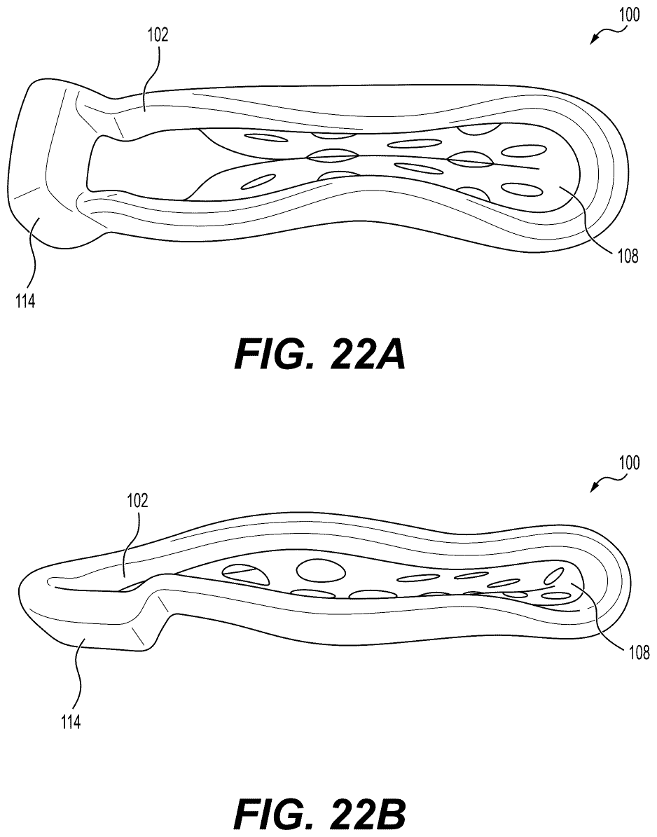

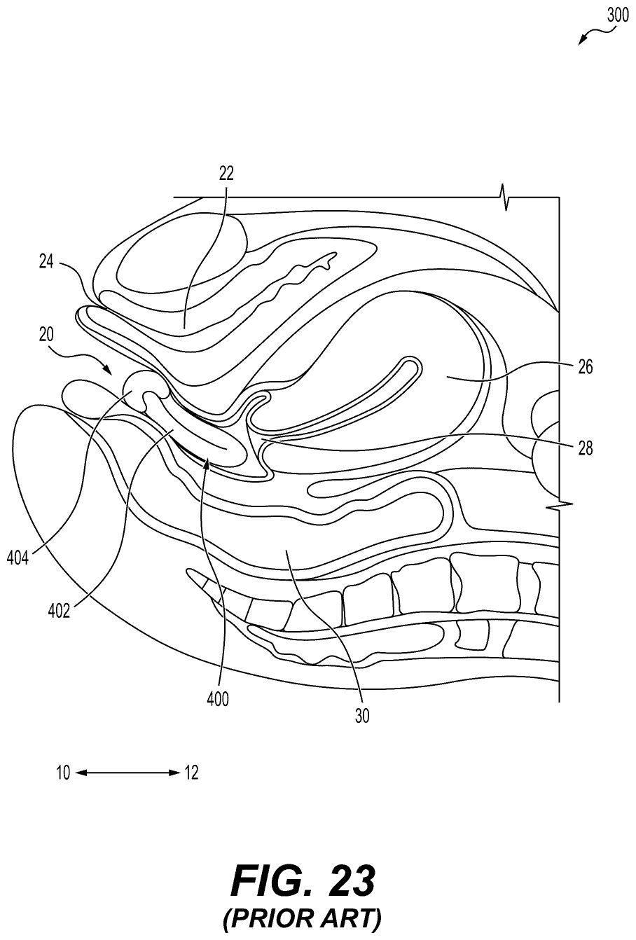

FIGS. 1-16 illustrate different views of an example pessary device 100 while in a deployed state, such as before any type of use or after the pessary device 100 has been delivered into a patient's vaginal canal. As further discussed below, pessary device 100 is configured to transition between a first position in a compressed state (e.g., used during insertion, FIGS. 22A-22B) and a second position in the deployed state such that the pessary device can be delivered into the patient's vaginal canal and placed transverse to the patient's vaginal canal when in the compressed state and then expand into the deployed state.

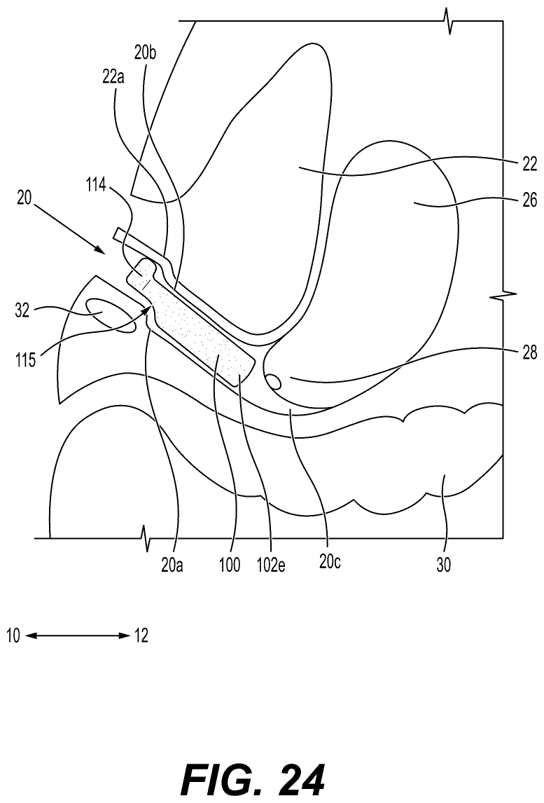

As shown throughout FIGS. 1-16, pessary device 100 is an intra-vaginal pessary device that can include an outer ring 102 that has an elliptical shape. The outer ring 102 includes a first opening 104. At least a portion of the outer ring 102 is configured to engage one or more vaginal surfaces of a patient. In these figures, the proximal direction is indicated by arrow 12 and the distal direction is indicated by arrow 10. For example, a proximal 12 surface 102e of the outer ring 102 may engage with the opening to the patient's cervix 28 (as particularly shown in FIG. 24).

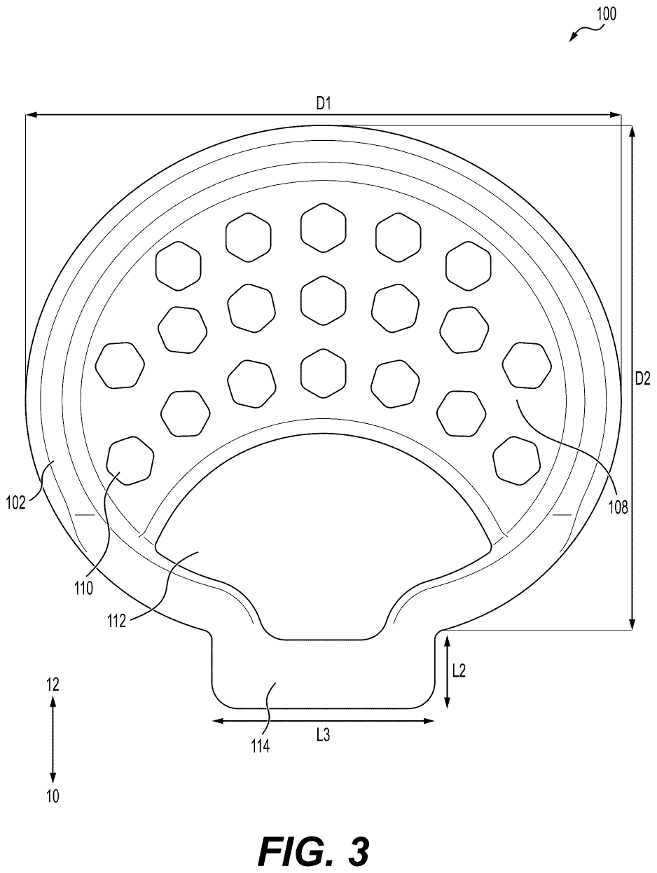

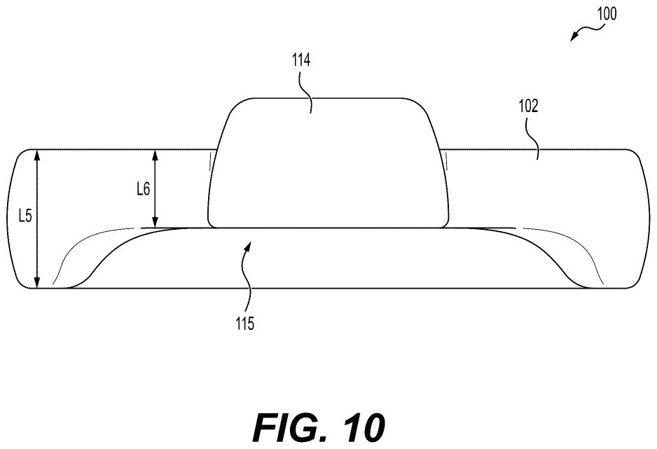

In some embodiments, as particularly shown in FIG. 3, a first diameter D1 of the outer ring 102 can be between approximately 55 to 71 millimeters (mm), while a second diameter D2 of the outer ring 102 can be between approximately 47 to 62 mm. In some embodiments, as particularly shown in FIG. 10, a first depth L5 of the outer ring 102 can be between approximately 13 to 17 mm, and a second depth L6 of the outer ring 102 can be between approximately 6 to 11 mm.

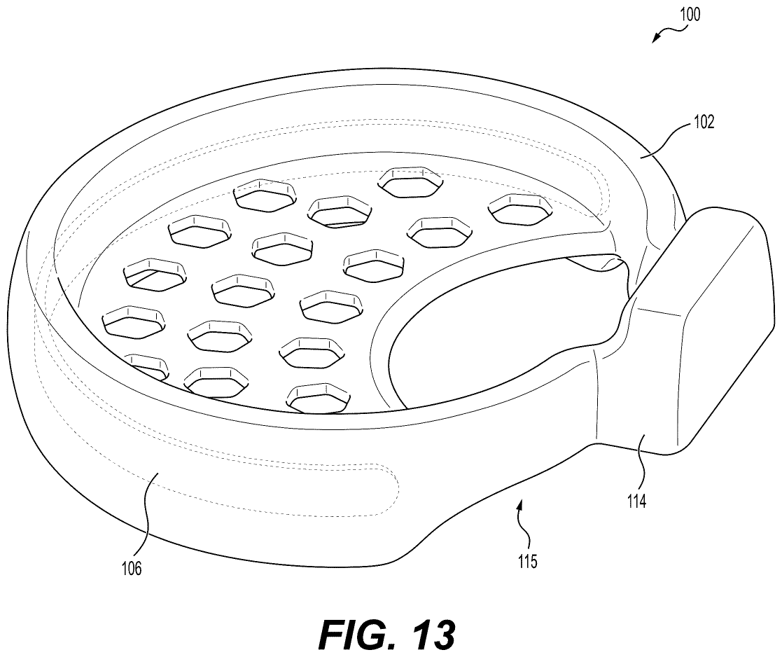

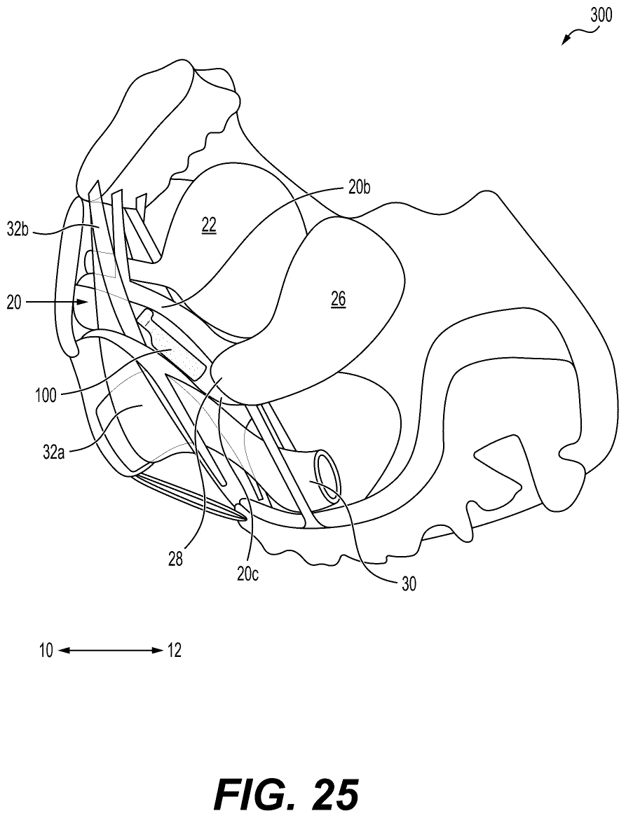

As particularly shown in FIGS. 13-16, the pessary device 100 can include a shape memory component 106, such as nickel titanium (or Nitinol), disposed within the outer ring 102 and internal to the pessary device. The shape memory component 106 is configured such that it becomes pliable when exposed to colder temperatures (e.g., below approximately 25 °C), and when heated (e.g., to above approximately 30 °C), reverts to its original shape. As such, inclusion of the shape memory component 106 within the outer ring 102 aids in the pliability of the pessary device 100. For example, the pessary device 100 can be cooled such that it is pliable enough to deliver into the patient's vaginal canal. Once delivered and exposed to the patient's internal body temperature, the pessary device 100 will attempt to revert to its original state, but will settle into a deployed state, as shown in FIG. 25, as the device 100 is at least partially restrained by the patient's vaginal canal.

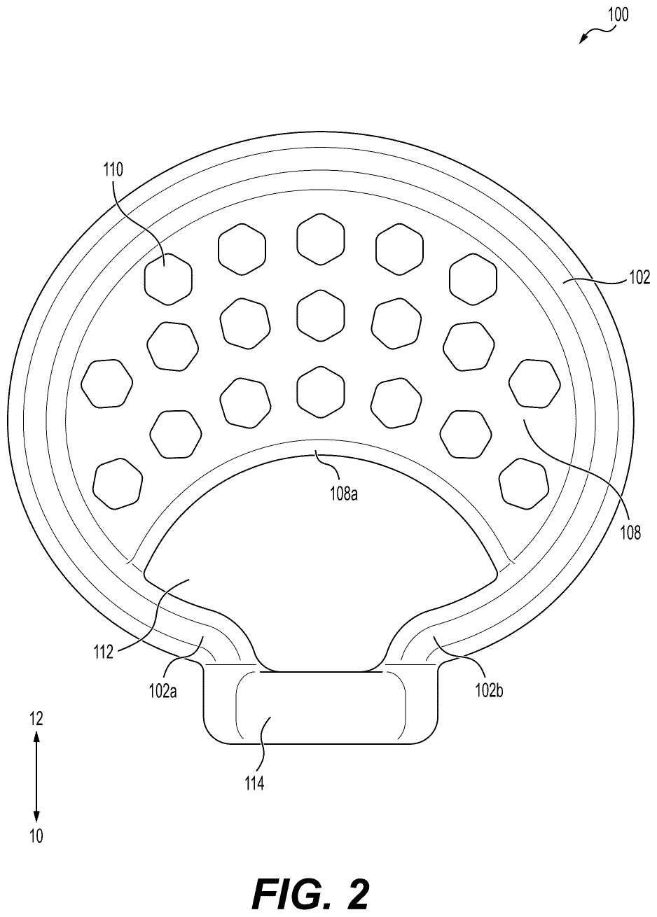

As particularly shown in FIGS. 1-3 and 12, pessary device 100 can include a net 108 disposed within the first opening 104 of the outer ring 102. The net 108 can have one or more second openings 110 configured to allow for drainage of fluid within the vaginal canal once the pessary device 100 is implanted. In some embodiments, as particularly shown in FIG. 2, at least a first edge 108a of the net 108 follows substantially the same curvature as at least a portion of the outer ring 102.

In some embodiments, as particularly shown in FIG. 1, the net 108 can be positioned between a top edge 102c of the outer ring 102 and a bottom edge 102d of the outer ring 102. In some embodiments, the net 108 can be equidistant from the top edge 102c and the bottom edge 102d of the outer ring 102.

As particularly shown in FIGS. 1-3, pessary device 100 can include a third opening 112 disposed within the first opening and distal 10 to the net 108. The third opening is configured to aid in delivering the pessary device into the patient's vaginal canal and/or removing the pessary device from the patient's vaginal canal. For example, the third opening 112 provides an opening through which a patient's or physician's finger could hold or grasp onto the pessary device 100 to make it easier to insert or remove the device from the patient's vaginal canal.

As particularly shown in FIGS. 1 and 4-5, pessary device 100 can include a distal knob 114 configured to provide urethral support. For example, as particularly shown in FIGS. 24-25, which illustrate how the pessary device 100 appears when positioned in a patient's vaginal canal, the distal knob 114 is configured to engage with an anterior wall of the vaginal canal that is itself adjacent to the urethra. As such, the distal knob 114 can provide support to the urethra if, for example, a patient is experiencing organ prolapse and/or incontinence.

As particularly shown in FIG. 2, the outer ring 102 can have a first end 102a and a second end 102b that are joined at the distal knob 114.

As particularly shown in FIG. 5, the distal knob 114 can extend at least a first distance L1 (e.g., between approximately 4 to 8 mm) above the outer ring 102. In some embodiments, as particularly shown in FIG. 3, a depth L2 of the distal knob 114 can be between approximately 6 to 10 mm, and a width L3 of the distal knob 114 can be between approximately 20 to 28 mm. In some embodiments, as particularly shown in FIG. 5, a height L4 of the distal knob 114 can be between approximately 12 to 16 mm.

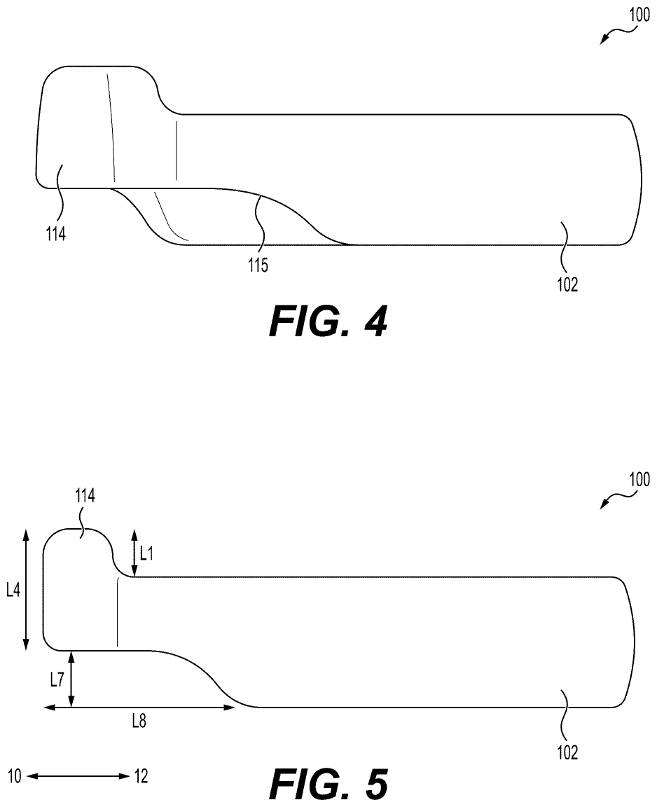

As particularly shown in FIGS. 4-5 and 10, pessary device 100 can include a cutout 115 disposed along the outer ring. As particularly shown in FIGS. 24-25, which illustrates how the pessary device 100 appears when positioned in a patient's vaginal canal, the cutout 115 is configured to engage the prominence of the posterior vaginal wall formed by one or more levator muscles of the patient. For example, the cutout 115 is configured to abut the posterior vaginal prominence formed from the puborectalis muscle (PRM) and the pubococcygeus muscle (PCM) underlying the posterior vaginal wall, thereby reducing distal migration of the device, reducing expulsion of the device, and/or ensuring that the device remains in situ to provide increased tissue support and/or comfort for the patient.

In some embodiments, as particularly shown in FIG. 5, a height L7 of the cutout 115 can be between approximately 3 to 9 mm, and a depth L8 of the cutout 115 can be between approximately 20 to 30 mm.

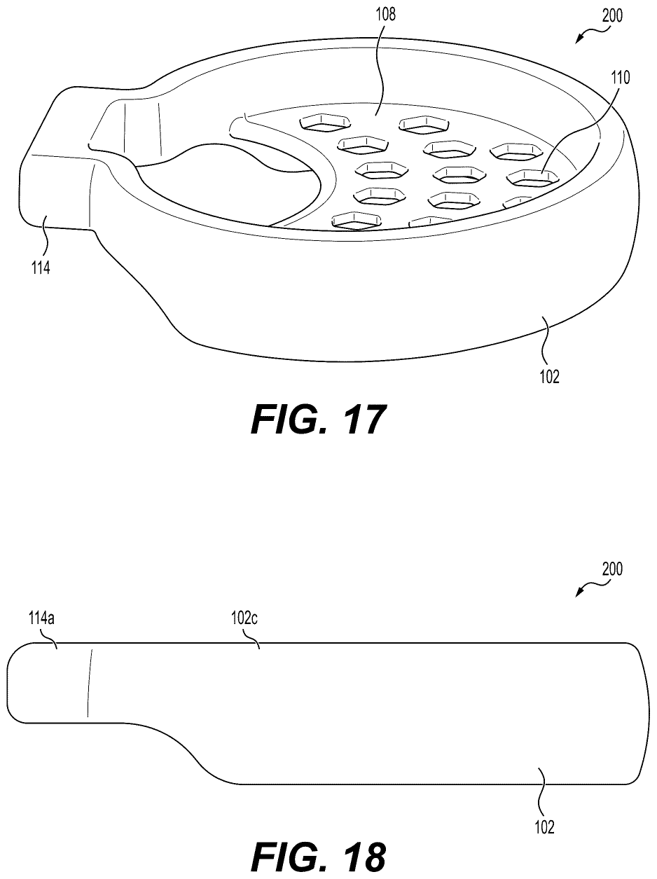

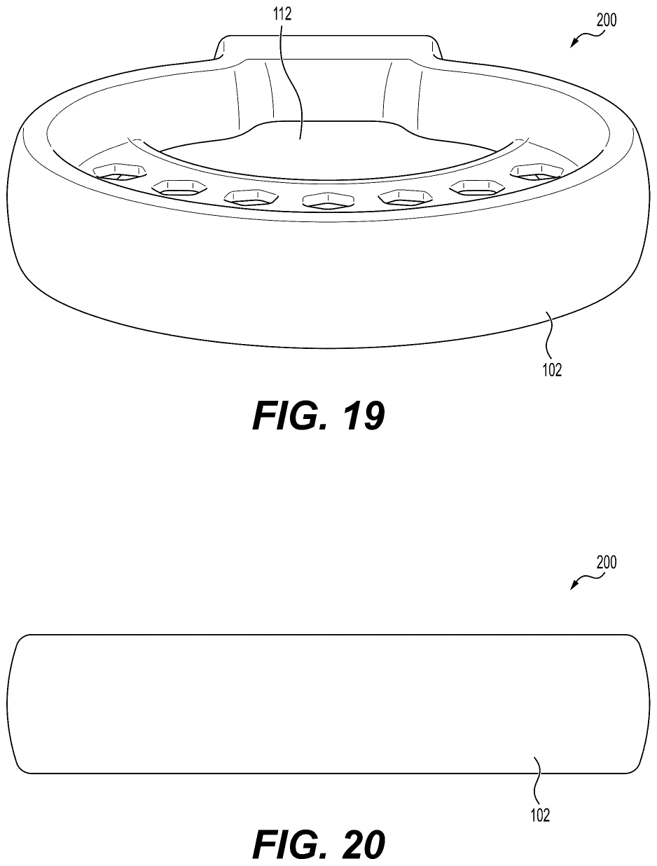

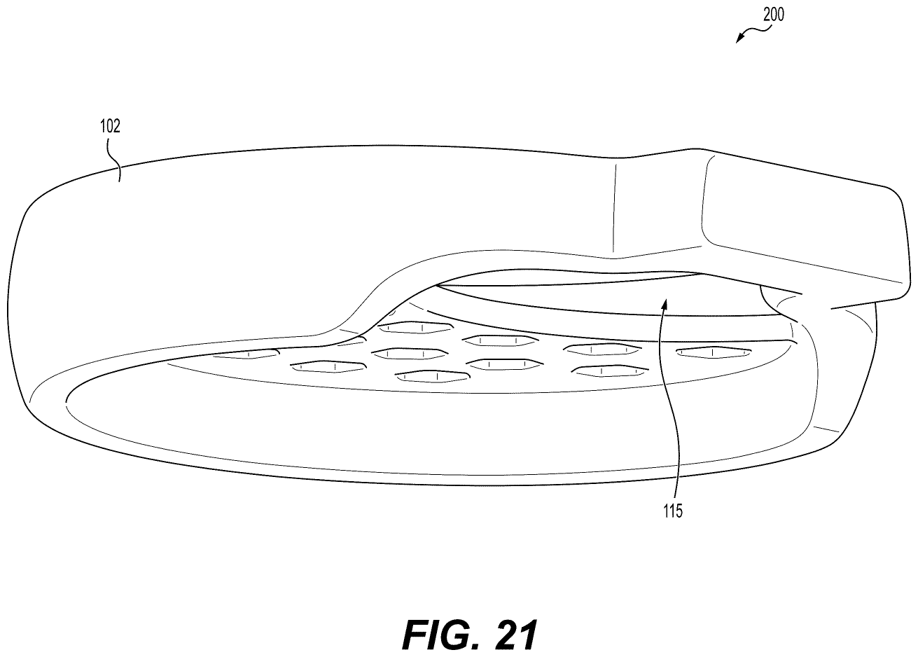

FIGS. 17-21 illustrate different views of another example pessary device 200 while in an original state. Pessary device 200 may function the same as or similarly to pessary device 100, as disclosed herein, and may have one or more of the same or similar components as pessary device 100. However, pessary device 200 may be configured such that its respective distal knob 114 does not extend above the top edge 102c of the outer ring 102. For example, as particularly shown in FIG. 18, a top edge 114a of the distal knob 114 can be aligned with the top edge 102c of the outer ring 102. A benefit of such alternative configuration is that pessary device 200 can be used in patients who might not need the amount of urethral support provided by the distal knob 114 of pessary device 100, for example, based on differing patient conditions, anatomical sizes, and/or patient comfort.

FIGS. 22A and 22B provide examples of how pessary device 100 (or pessary device 200) can be manipulated into a compressed state. Once in such compressed state, the pessary device can be inserted or delivered into the patient's vaginal canal. Once in the proper position (e.g., placed transverse to the patient's vaginal canal, or placed laterally within the patient's vaginal canal), the pessary device can be left in place and will transition to a deployed state to remain in the vaginal canal, as discussed herein, until the next time a user (e.g., a patient or physician) needs to manipulate the device (e.g., remove).

FIGS. 23-25 provide comparisons between how existing pessary devices (FIG. 23) versus the disclosed pessary devices (FIGS. 24-25) are positioned within the vaginal canal.

FIG. 23 shows a system 300 including an existing pessary device 400 placed in a patient's vaginal canal 20. Other surrounding anatomy includes the bladder 22, urethra 24, uterus 26, cervix 28, and rectum 30. As shown, pessary device 400, similar to many existing pessary devices, includes a ring 402, optionally with a knob 404 on a distal 10 end of the ring 402. Given its shape and configuration, pessary device 400, like other existing pessary devices, is more prone to spontaneous displacement or rotation, rendering it less effective (e.g., POP and SUI) and more likely to be expelled from proper placement in the vaginal canal.

FIGS. 24-25 show how the disclosed pessary device 100 can alternatively be positioned within system 300 to overcome challenges faced with existing pessary devices. For example, pessary device 100 can be positioned within the vaginal canal 20 such that the cutout 115 engages with one or more levator muscles (e.g., 32a, 32b) of the patient. For example, the vagina and rectum of a female are encircled by the PRM which, in turn, is medial to the PCM. The cutout 115 is configured to abut the posterior vaginal prominence formed from the PRM 32a and PCM 32b underlying the posterior vaginal wall 20a, thereby reducing distal migration of the device, reducing expulsion of the device, and/or ensuring that the device remains in situ to provide increased tissue support and/or comfort for the patient. Pessary device 100 also engages with the vaginal posterior wall 20a and the vaginal anterior wall 20b, and is situated within the vaginal canal 20 such that it aligns distally within the region of mid urethra to the neck of the bladder 22a. For example, inclusion of the cutout 115 enables the posterior aspect of pessary device 100 (or 200) to position the distal anterior aspect of the device, to thereby support the urethra. In contrast, existing pessary devices, such as existing pessary device 400 (FIG. 23), can migrate thereby providing less consistent support of the urethra. Additionally, as particularly shown in FIG. 24, the pessary device 100 is positioned such that the cervix 28 faces the net 108 of the device 100 (or first opening of the outer ring if no net is included), and the proximal surface 102e of the outer ring is positioned at or around the posterior vaginal fornix 20c. It should be understood that either pessary device 100 or pessary device 200 could be similarly used in system 300 to provide one or more of the benefits discussed herein.

The disclosed technology described herein can be further understood according to the following clauses.

Clause 1: An intra-vaginal pessary device (100) comprising: an outer ring (102) having an elliptical shape and comprising a first opening (104), wherein at least a portion of the outer ring is configured to engage one or more vaginal surfaces of a patient; a shape memory component (106) disposed within the outer ring (102) and internal to the pessary device; a net (108) disposed within the first opening of the outer ring, the net comprising one or more second openings (110); a third opening (112) disposed within the first opening and distal (10) to the net, the third opening configured to aid in at least removing the pessary device from the patient's vaginal canal; a distal knob (114) configured to provide urethral support; and a cutout (115) disposed along the outer ring and configured to engage one or more levator muscles of the patient, wherein the pessary device is movable between a first position in a compressed state and a second position in a deployed state such that the pessary device can be delivered into the patient's vaginal canal and placed transverse to the patient's vaginal canal when in the compressed state and then expand into the deployed state.

Clause 2: The pessary device of Clause 1, wherein the pessary device comprises silicone.

Clause 3: The pessary device of any of Clauses 1-2, wherein the shape memory component comprises Nitinol.

Clause 4: The pessary device of any of Clauses 1-3, wherein at least a first edge (108 a) of the net (108) follows substantially the same curvature as at least a portion of the outer ring (102).

Clause 5: The pessary device of any of Clauses 1-4, wherein the distal knob (114) extends at least a first distance (L1) above the outer ring (102).

Clause 6: The pessary device of Clause 5, wherein the first distance (L1) is between approximately 4 to 8 millimeters.

Clause 7: The pessary device of any of Clauses 1-6, wherein: the outer ring (102) comprises a first end (102a) and a second end (102b), and the first and second ends (102a, 102b) are joined via the distal knob (114).

Clause 8: The pessary device of any of Clauses 1-7, wherein the net (108) is between a top edge (102c) of the outer ring (102) and a bottom edge (102d) of the outer ring (102).

Clause 9: The pessary device of any of Clauses 1-8, wherein a first diameter (D1) of the outer ring (102) is between approximately 55 to 71 mm.

Clause 10: The pessary device of any of Clauses 1-9, wherein a second diameter (D2) of the outer ring (102) is between approximately 47 to 62 mm.

Clause 11: The pessary device of any of Clauses 1-10, wherein a depth (L2) of the distal knob (114) is between approximately 6 to 10 mm, and wherein a width (L3) of the distal knob (114) is between approximately 20 to 28 mm.

Clause 12: The pessary device of any of Clauses 1-11, wherein a height (L4) of the distal knob (114) is between approximately 12 to 16 mm.

Clause 13: The pessary device of any of Clauses 1-12, wherein a height (L7) of the cutout (115) is between approximately 3 to 9 mm, and wherein a depth (L8) of the cutout (115) is between approximately 20 to 30 mm.

Clause 14: The pessary device of any of Clauses 1-13, wherein: a first depth (L5) of the outer ring (102) is between approximately 13 to 17 mm, and a second depth (L6) of the outer ring (102) is between approximately 6 to 11 mm.

Clause 15: An intra-vaginal pessary device (100) comprising: an outer ring (102) having an elliptical shape and comprising a first opening (104), wherein at least a portion of the outer ring is configured to engage one or more vaginal surfaces of a patient; a shape memory component (106) disposed within the outer ring (102) and internal to the pessary device; a second opening (112) disposed within the first opening (104), the second opening configured to aid in at least removing the pessary device from the patient's vaginal canal; and a cutout (115) disposed along the outer ring and configured to engage one or more levator muscles of the patient, wherein the pessary device is movable between a first position in a compressed state and a second position in a deployed state such that the pessary device can be placed transversely into the patient's vaginal canal when in the compressed state and then expand into the deployed state

Clause 16: The pessary device of Clause 15, further comprising: a distal knob (114), wherein: the outer ring (102) comprises a first end (102a) and a second end (102b), the first and second ends (102a, 102b) are joined via the distal knob (114), and wherein a first top edge (114a) of the distal knob (114) is aligned with a second top edge (102c) of the outer ring (102).

Clause 17: An intra-vaginal pessary device (100) comprising: an outer ring (102) configured to engage one or more vaginal surfaces of a patient; and a cutout (115) disposed along the outer ring (102) and configured to engage one or more levator muscles of the patient when the pessary device is placed in a deployed state within the patient's vaginal canal, wherein the pessary device is movable between a first position in a compressed state and a second position in a deployed state such that the pessary device can be placed within the patient's vaginal canal.

Clause 18: The pessary device of Clause 17, wherein: the one or more levator muscles comprise the puborectalis muscle (PRM) and the pubococcygeus muscle (PCM), the PRM and the PCM form a posterior vaginal prominence underlying a posterior vaginal wall, and the cutout is configured to abut the posterior vaginal prominence.

Clause 19: The pessary device of any of Clauses 17-18, wherein the cutout is configured to reduce distal migration of the pessary device within the patient's vaginal canal, reduce a chance of expulsion of the pessary device from within the patient's vaginal canal, and aid in the pessary device remaining in situ thereby providing increased tissue support.

Clause 20: The pessary device of any of Clauses 17-19, further comprising: a net (108) disposed within the outer ring (102) and comprising one or more second openings (110), wherein the net (108) is equidistant from a top edge (102c) of the outer ring (102) and a bottom edge (102d) of the outer ring (102).

What is claimed is:

1. An intra-vaginal pessary device comprising:
an outer ring having an elliptical shape and comprising a first opening, wherein at least a portion of the outer ring is configured to engage one or more vaginal surfaces of a patient;
a shape memory component disposed within the outer ring and internal to the pessary device;
a net disposed within the first opening of the outer ring, the net comprising one or more second openings;
a third opening disposed within the first opening and distal to the net, the third opening configured to aid in at least removing the pessary device from the patient's vaginal canal;
a distal knob configured to provide urethral support; and
a cutout disposed along the outer ring and configured to engage one or more levator muscles of the patient,
wherein the pessary device is movable between a first position in a compressed state and a second position in a deployed state such that the pessary device can be delivered into the patient's vaginal canal and placed transverse to the patient's vaginal canal when in the compressed state and then expand into the deployed state.

2. The pessary device of claim 1, wherein the pessary device comprises silicone.

3. The pessary device of claim 1, wherein the shape memory component comprises Nitinol.

4. The pessary device of claim 1, wherein at least a first edge of the net follows substantially the same curvature as at least a portion of the outer ring.

5. The pessary device of claim 1, wherein the distal knob extends at least a first distance above the outer ring.

6. The pessary device of claim 5, wherein the first distance is between approximately 4 to 8 millimeters.

7. The pessary device of claim 1, wherein:
the outer ring comprises a first end and a second end, and the first and second ends are joined via the distal knob.

8. The pessary device of claim 1, wherein the net is between a top edge of the outer ring and a bottom edge of the outer ring.

9. The pessary device of claim 1, wherein a first diameter of the outer ring is between approximately 55 to 71 mm.

10. The pessary device of claim 1, wherein a second diameter of the outer ring is between approximately 47 to 62 mm.

11. The pessary device of claim 1, wherein a depth of the distal knob is between approximately 6 to 10 mm, and wherein a width of the distal knob is between approximately 20 to 28 mm.

12. The pessary device of claim 1, wherein a height of the distal knob is between approximately 12 to 16 mm.

13. The pessary device of claim 1, wherein a height of the cutout is between approximately 3 to 9 mm, and wherein a depth of the cutout is between approximately 20 to 30 mm.

14. The pessary device of claim 1, wherein:
a first depth of the outer ring is between approximately 13 to 17 mm, and
a second depth of the outer ring is between approximately 6 to 11 mm.

15. An intra-vaginal pessary device comprising:
an outer ring having an elliptical shape and comprising a first opening, wherein at least a portion of the outer ring is configured to engage one or more vaginal surfaces of a patient;
a shape memory component disposed within the outer ring and internal to the pessary device;
a second opening disposed within the first opening, the second opening configured to aid in at least removing the pessary device from the patient's vaginal canal; and
a cutout disposed along the outer ring and configured to engage one or more levator muscles of the patient,
wherein the pessary device is movable between a first position in a compressed state and a second position in a deployed state such that the pessary device can be placed transversely into the patient's vaginal canal when in the compressed state and then expand into the deployed state.

16. The pessary device of claim 15, further comprising:
a distal knob,
wherein:
the outer ring comprises a first end and a second end,
the first and second ends are joined via the distal knob, and
wherein a first top edge of the distal knob is aligned with a second top edge of the outer ring.

17. An intra-vaginal pessary device comprising:
an outer ring configured to engage one or more vaginal surfaces of a patient; and
a cutout disposed along the outer ring and configured to engage one or more levator muscles of the patient when the pessary device is placed in a deployed state within the patient's vaginal canal,
wherein the pessary device is movable between a first position in a compressed state and a second position in a deployed state such that the pessary device can be placed within the patient's vaginal canal.

18. The pessary device of claim 17, wherein:
the one or more levator muscles comprise the puborectalis muscle (PRM) and the pubococcygeus muscle (PCM),
the PRM and the PCM form a posterior vaginal prominence underlying a posterior vaginal wall, and
the cutout is configured to abut the posterior vaginal prominence.

19. The pessary device of claim 18, wherein the cutout is configured to reduce distal migration of the pessary device within the patient's vaginal canal, reduce a chance of expulsion of the pessary device from within the patient's vaginal canal, and aid in the pessary device remaining in situ thereby providing increased tissue support.

20. The pessary device of claim 17, further comprising:
a net disposed within the outer ring and comprising one or more second openings,
wherein the net is equidistant from a top edge of the outer ring and a bottom edge of the outer ring.
